(19) United States
(12) Patent Application Publication
(10) Pub. No.: US 2012/0097884 A1
(43) Pub. Date: Apr. 26, 2012

(54) USE OF AEROGELS FOR PREPARING A MATERIAL FOR THERMAL INSULATION

(75) Inventors: Brigitte Jamart-Gregoire, Nicolas (FR); Nicolas Brosse, Vezelise (FR); Quoc Nghi Pham, Le Mans (FR); Danielle Barth, Ludres (FR); Alexandre Scondo, Tomblaine (FR); Alain Degiovanni, Nancy (FR)

(73) Assignee: INSTITUT NATIONAL POLYTECHNIQUE DE LORRAINE, Vandoeuvre Les Nancy Cedex (FR)

(21) Appl. No.: 13/320,800

(22) PCT Filed: May 18, 2010
(86) PCT No.: PCT/FR10/50959

§ 371 Date: Jan. 9, 2012

(30) Foreign Application Priority Data
May 20, 2009 (FR) 0953363

(51) Int. Cl. E04B 1/78; C07D 221/06

(52) U.S. Cl. 252/62; 546/99

(57) ABSTRACT

Material for thermal insulation including an aerogel obtained by drying an organogel prepared from the pseudopeptides of formula (I).

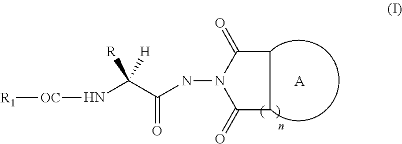

in which R represents a side chain of an amino acid, R₁ represents a (C₁-C₈)alkyl, (C₁-C₈)alkoxy, aryl, aryloxy, or glycoside group, n=1 or 2 and A represents an aromatic group with one or more rings.

The invention relates to a material for thermal insulation prepared from aerogels.

Thermal insulation, encompassing methods used to limit heat transfers between a hot medium and a cold medium, is used in particular in the construction, industrial and automotive fields.

The materials used are very varied and there can be mentioned in particular synthetic materials (expanded and extruded polystyrenes, polyurethane, polyester), mineral, vegetable and animal fibres, (rockwools, glass wools; wood, flax, hemp, sheep's wools etc.), other renewable materials (cellulose, cork, etc.), more rarely used mineral insulating materials (perlite, vermiculite, expanded clay, cellular glass) and thin reflective insulating materials.

More recently aerogels (a material similar to a gel where the liquid component is replaced by gas) have been employed in construction and in consumer products, such as sleeping bags and gloves, tennis rackets, etc.

Aerogels are defined as dry gels generally having pores of nanometric volume. This type of material is obtained by the supercritical drying of organogels which makes it possible to eliminate the solvent while retaining the porous texture of the liquid gel. The aerogels described in the literature are obtained from varied and diverse polymer structures or from oxides such as alumina or silica. The most widely-used silica aerogels are constituted by microbeads of a porous glass based on amorphous silicon dioxide.

However, these aerogels are still costly and there is a need to make new aerogels available that have even higher performance and the manufacture of which is less costly.

The inventors have recently discovered fortuitously that a series of compounds of low molecular weight derived from natural amino acids were capable of gelling apolar solvents even at very low concentrations. These compounds are described by Brosse N. et al. (Tetrahedron Letters, 45, (2004) 9521-9524). The aerogels obtained from these organogels by evaporation of the solvent are mesoporous nanostructured materials which have remarkable properties, in particular in terms of specific surface area and very low solid contribution, making it possible to envisage the use of these materials in various applications, in particular as insulators, catalysts, thickeners for paint or cosmetics, etc.

Thus the invention relates to a material for thermal insulation comprising an aerogel obtained by drying an organogel, said organogel having been prepared from pseudopeptides of formula (I)

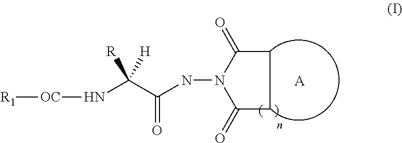

in which
R represents a side chain of a natural or synthetic amino acid,
R₁ represents either a linear or branched (C₁-C₈)alkyl group, or a linear or branched (C ₁-C₈)alkoxy group, or an aryl group, or an aryl(C₁-C₄)alkyl group, or an aryloxy group, or a saturated or unsaturated glycoside,
n=1 or 2 and
A represents an aromatic or heteroaromatic group with one or more rings, in particular a phenyl group or a naphthyl group.

In an advantageous embodiment of the material for thermal insulation according to the invention, the pseudopeptide of formula (I) is chosen from those in which the

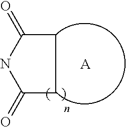

group represents either a

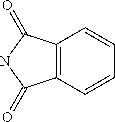

group or a

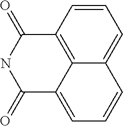

group.

In an even more advantageous embodiment of the invention, the pseudopeptide of formula (I) is chosen from those in which

R represents either —CH₂Ph, or —CH(CH₃)₃ or —CH₂CH(CH₃)₃ and
R₁ represents either PhCH₂OCO—, or CH₂=CH—CH₂OCO—.

Within the meaning of the present invention, by linear or branched (C₁-C₈)alkyl is meant a hydrocarbon chain with 1 to 12 carbon atoms, in particular with 1 to 6 carbon atoms. Such as for example the following groups: methyl, ethyl, propyl, isopropyl, butyl, isobutyl, sec-butyl, tert-butyl, pentyl, isopentyl, neopentyl, tert-pentyl, hexyl, isohexyl, 1,1-dimethylbutyl, 2,2-dimethylbutyl, 3,3- dimethylbutyl and 2-ethylbutyl.

Within the meaning of the present invention, by natural or synthetic amino acid is meant in particular the following amino acids: aspartic acid (Asp or D), asparagine (Asn or N), threonine (Thr or T), serine (Ser or S), glutamic acid (Glu or E), glutamine (Gln or Q), glycine (Gly or G), alanine (Ala or A), cysteine (Cys or C), valine (Val or V), methionine (Met or M), isoleucine (Ile or I), leucine (Leu or L), tyrosine (Tyr or Y), phenylalanine (Phe or F), histidine (His or H), lysine (Lys or K), tryptophan (Trp or W), proline (Pro or P) and arginine (Arg or R).

Within the meaning of the present invention, by aryl is meant a group chosen from phenyl, benzyl, tolyl, xylyl and naphthyl.

Within the meaning of the present invention, by saturated or unsaturated heterocycle or heteroaromatic is meant a group chosen from oxiranyl, azetidinyl, oxetanyl, thietanyl, pyrrolidinyl, tetrahydrofuryl, thiolanyl, piperidyl, tetrahydropyranyl, morpholinyl, thiomorpholinyl and piperazinyl, pyridyl, pyrimidinyl, pyridazinyl, pyrrolyl, furanyl, thiophenyl, imidazolyl, pyrazolyl, isoxazolyl, isothiazolyl, thiazolyl, oxazolyl, 1,2,4-oxadiazolyl, 1,2,3-oxadiazolyl, 1,3,4-oxadiazolyl, 1,2,3-thiadiazolyl, 1,2,4-thiadiazolyl, 1,3,4-thiadiazolyl, 1,2,4-triazolyl, 1,3,4-triazolyl, 1,2,3-triazolyl and tetrazolyl; benzofuranyl, isobenzofuranyl, benzo[b]thienyl, indolyl, isoindolyl, 1H-indazolyl, benzimidazolyl, benzoxazolyl, benzisoxazolyl, benzothiazolyl, benzisothiazolyl, benzodioxolyl, 1H-benzotriazolyl, quinoline, isoquinolyl, cinnolinyl, quinazolinyl, quinoxalinyl and phthalazinyl.

The pseudopeptides of formula (I) can be synthesized in three stages from natural or synthetic amino acids and inexpensive commercial reagents following standard techniques known to a person skilled in the art or described in the literature.

The organogels can also be obtained following standard techniques known to a person skilled in the art or described in the literature. By way of example, they can be prepared by heating a pseudopeptide of formula (I) to reflux in a solvent such as for example carbon tetrachloride, in proportions comprised between 0.01 and 5%, advantageously between 0.2 and 2% by weight of organogelators with respect to the solvents, followed by cooling.

The aerogels are prepared by drying organogels in a supercritical CO₂ medium by standard techniques known to a person skilled in the art or described in the literature, such as for example that described in French patent FR2584618.

The aerogels used according to the invention have a specific surface area greater than 50 m²/g measured by the B.E.T. method by nitrogen adsorption, an average pore diameter of 35.3 A and a thermal conductivity less than 0.03 W·m⁻¹·K⁻¹ at atmospheric pressure and less than 0.003 W·m⁻¹·K⁻¹ under vacuum.

A further subject of the present invention is the use for the preparation of a material for thermal insulation of an aerogel obtained by drying an organogel, said organogel having been prepared from the pseudopeptides of formula (I).

The material for thermal insulation according to the invention can moreover comprise additives chosen from binders, ionic compounds, opacifying agents and fibres.

It can be presented in all the usual forms and in particular in the form of slabs, blocks.

It can be used in all the usual applications for this type of material, in particular in the field of thermal super-insulators, in particular under vacuum in industrial freezers or refrigerators.

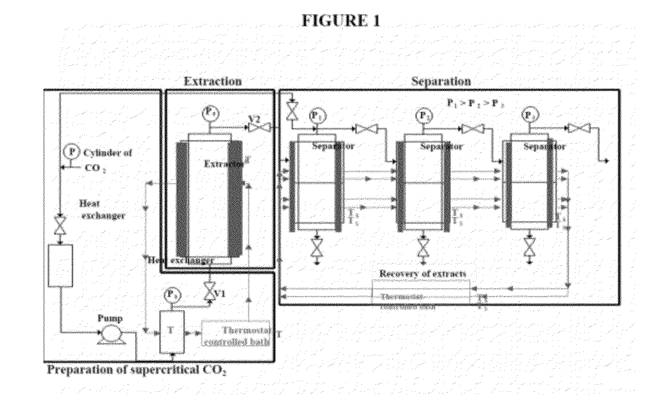

Example 1 and FIGS. 1 to 4 below illustrate the invention.

FIG. 1 is a diagram of the dynamic drying process.

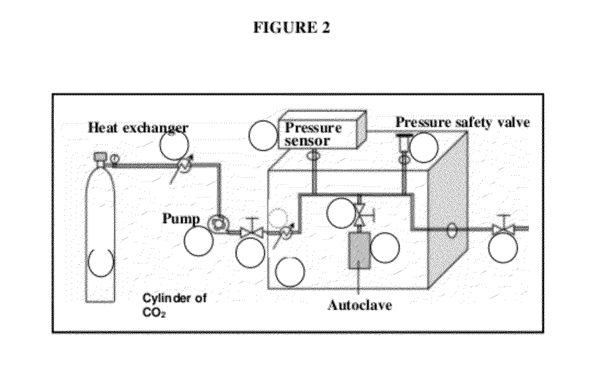

FIG. 2 is a diagram of the supercritical CO₂ drying process.

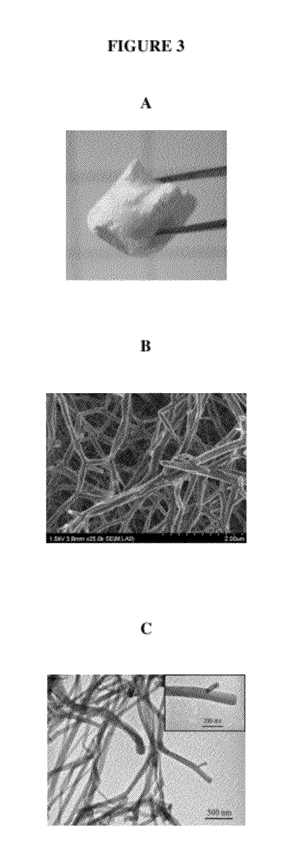

FIG. 3 shows (a) a photograph of an aerogel sample prepared according to the example by drying following the second process; (b) an image obtained by Scanning Electron Microscopy of said sample, and (c) a TEM image of said sample.

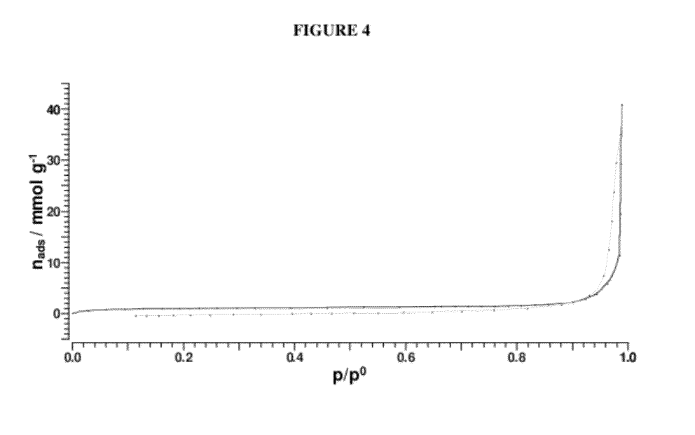

FIG. 4 shows the nitrogen adsorption isotherm of an aerogel sample obtained by drying organogel at 2% weight.

### EXAMPLE 1

### Preparation of an Aerogel from the Compound of Formula (Ia)

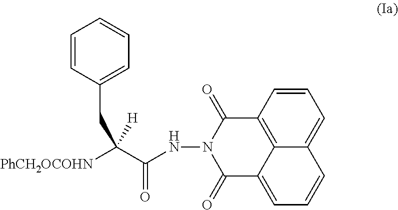

### Preparation of Compound (Ia)

L-Phenylalanine methyl ester hydrochloride (10.75 g, 50 mmol) is dissolved in a saturated solution of NaHCO₃ (200 mL) and chlorobenzyl carbonate (8.5 g, 50 mmol) is added under vigorous stirring overnight. The solution is extracted with ether (3 times). The organic phases are washed with a 1N solution of hydrochloric acid, dried by the addition of MgSO₄ and concentrated by evaporating the solvents. The excess chlorobenzyl carbonate is eliminated by passing the crude reaction product over a small silica column, the eluent used being petroleum ether. The product is then chromatographed with an eluent 40/60 EtOAc/petroleum ether and leads to 14 g (90%) N-benzyloxycarbonyl-L-phenylalanine methyl ester in the form of a pure product.

Hydrazine hydrate (5 g, 100 mmol) is added to a solution of N-benzyloxycarbonyl-L-phenylalanine methyl ester (10 g, 32 mmol) in methanol (100 mL). The mixture is stirred overnight at ambient temperature and the hydrazide formed is collected by filtration, washed with methanol and dried. (7.8 g, 78%).

Hydrazide (2 g, 6.3 mmol) is added to a suspension of naphthalic anhydride (1.26 g, 6.3 mmol) in toluene (200 mL) and the mixture is taken to reflux. The water formed during the reaction is trapped by a Dean-Stark apparatus. After 6 hours, the cooled solution rapidly transforms into a gelatinous mass which after evaporation of the solvents leads to a solid. The solid is then recrystallized from chloroform.

Compound Ia was identified by proton NMR proton NMR (300 MHz, CDC1₃) of Ia: ˜8.85 (s, 1H), 8.65-8.45 (m, 2H), 8.20 (d, 2H), 7.80-7.60 (m, 2H), 7.45-7.10 (m, 1OH), 5.63 (m, 1H), 5.15-5.00 (m, 2H), 5.00-4.75 (m, 1H), 3.38 (dd, 1H), 3.18 (dd, 1H).

### 1.1. Obtaining the Aerogels

### 1.1.1. Preparation of the Gel

17.4 mg of compound (Ia) was dissolved in 2 mL toluene in order to obtain a gel at 1% by weight.

### 1.1.2. Drying the Gel.

### 1.2.2.1. Old Drying System (Dynamic Extraction):

The system is shown in FIG. 1
Preparation of the drying cell: the gel was placed in the drying cell (dimensions of the autoclave: φᵢ=23 mm, h=300 mm, V=125 cm³). The two ends were closed by sintered metal discs. Before placing the cell in the autoclave, 2 mL toluene was previously added. Then a further 2 mL toluene was added in the cell. Adding toluene before and after the gel is intended to avoid spontaneous evaporation of the solvent in the gel when the CO₂ enters the autoclave. The total volume of toluene is approximately 6 mL. The effective volume of the autoclave is approximately 81.3 mL.

The CO₂ gas is liquefied, then it passes through a pump having a metal diaphragm which regulates the flow (Dosapro Milton Roy-MilRoyal D, maximum flow rate 3.2 Kg·s⁻¹). After heating the CO₂ is ready to enter the extraction reactor under critical phase. The flow of CO₂ is measured at the entrance by a flowmeter (Micro Motion). The CO₂ is pumped into the autoclave until reaching the set pressure of 90 bars, while maintaining T_{extractor} at 15° C. After homogenization (5 minutes), the valves are opened and adjusted in order to obtain pressures P₁ (1^{st} separator) at 60 bars, P₂ (2^{nd} separator) at 45 bars and P₃ (3^{rd} separator) at 20 bars. The separators are respectively at a temperature of 20° C., 25° C. and 25° C. The circulation of the CO₂ is maintained for 15 minutes at a flow rate of approximately of 300-400 g/h. The outlet valve of the extraction autoclave is then closed and the temperature of the autoclave is taken to 45° C. The pressure of the extractor reaches 110-116 bars. The set pressure is taken to this value and the CO₂ circulates for 1 hour for the experiment at a flow rate of approximately 250 g/h. During this period, the toluene of the separators is sampled every 15 minutes.

### 1.2.2.2 New Drying System (Supercritical Reactor)

The main difference with the old system is the internal diameter of the reactor. It is 4 cm instead of 1.9 cm (dimensions of the reactor: h=8 cm, V=100 ml.). Moreover, for separating the toluene, this installation has only a single separator at 3° C. (instead of 3 separators in the previous model). The system is shown in FIG. 2.

The gel or the gel+cell system for measuring thermal conduction is placed in the reactor. 2 mL toluene was added in order to avoid spontaneous evaporation of solvent in the gel when the CO₂ enters the reactor. When in addition to the organogel, a cell for measuring thermal conduction is introduced, its position is fixed by adding glass beads. The liquid CO₂ is then pumped into the reactor until reaching the set pressure of 90 bars, while maintaining T_{reactor} at 15° C. After a homogenization phase of 5 minutes, the outlet valve is opened and adjusted so as to obtain the pressure P₁ (separator column) at 20 bars. The temperature of the separation column is maintained at 3° C. The circulation of the CO₂ is maintained for 10 minutes at a flow rate of approximately 400 g/h. The temperature of the reactor is then taken to 45° C. The CO₂ then circulates for 2 h at a rate of approximately 400 g/h and the toluene is sampled from the separators every 15 minutes.

### 1.1.3. Characteristics of the Aerogels Obtained

These are given in FIGS. 3 to 4.

The results obtained by MEB and MET show that the aerogels obtained are constituted by fibres having average diameters between 25-200 nm and micrometric length. It can also be observed that these materials are very porous (FIG. 3).

The basic characteristics of these aerogel nanomaterials are as follows:
1. Density of the backbone: 1.346±0.002 g/cm³ (helium pycnometry)
2. Density: ˜2.83 10⁻³ g/cm³
3. Specific surface area (SSA): 90.5 m²/g (B.E.T with nitrogen adsorption)
4. Cumulative volume of the pores: 1.482 cm²/g
5. Average pore diameter: 35.3 Å determined by the method of B.C. Lippens et al. J. Catalysis (1964), 3, 32.

At atmospheric pressure the thermal conductivity measured by the Flash method (A. Degiovanni Diffusivité et Methode Flash Revue générale de thermique n° 185 pp 417-442 May 1997) is close to air and of the order of 0.005 Wm⁻¹K⁻¹ under vacuum. Moreover, the very hydrophobic nature of the product prevents re-uptake of moisture, which gives the product a thermal stability over time. Thus placed in a beaker filled with water, the aerogel rapidly moves towards the walls of the beaker in order to avoid contact with water as much as possible.

1-8. (canceled)

9. Material for thermal insulation comprising an aerogel obtained by drying an organogel, said organogel having been prepared from the pseudopeptides of formula (I). 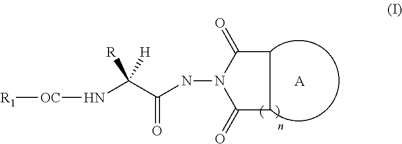
in which
R represents a side chain of a natural or synthetic amino acid,
R₁ represents either a linear or branched (C₁-C₈)alkyl group, i.e. a linear or branched (C₁-C₈ )alkoxy group, or an aryl group, or an aryl(C₁-C₄)alkyl group, or an aryloxy group, or a saturated or unsaturated heterocycle,
n=1 or 2 and
A represents an aromatic or heteroaromatic group with one or more rings.

10. Material for thermal insulation according to claim 9, characterized in that the pseudopeptide of formula (I) is chosen from those in which the 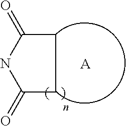
group represents either a 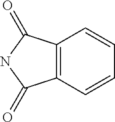
group or a 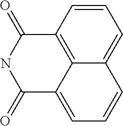
group.

11. Material for thermal insulation according to claim 9, characterized in that the pseudopeptide of formula (I) is chosen from those in which R represents either —CH₂Ph, or —CH(CH₃)₃ or —CH₂CH(CH₃)₃ and R₁ represents either PhCH₂OCO—, or CH₂═CH—CH₂OCO—.

12. Material for thermal insulation according to claim 9, characterized in that it moreover comprises additives chosen from binders, ionic compounds, opacifying agents and fibres.

13. Material for thermal insulation according to claim 9, characterized in that the aerogel has a specific surface area greater than 50 m²/g measured by B.E.T. by nitrogen absorption.

14. Material for thermal insulation according to claim 9, characterized in that the aerogel has an average pore diameter of 35.3 Å.

15. Material for thermal insulation according to claim 9, characterized in that the aerogel has a thermal conductivity less than 0.03 W·m⁻¹·K⁻¹ at atmospheric pressure and less than 0.003 W·m⁻¹·K⁻¹ under vacuum.

16. Use for the preparation of a material for thermal insulation of an aerogel obtained by drying an organogel, said organogel having been prepared from the pseudopeptides of formula (I) 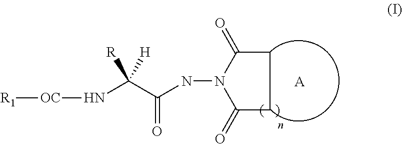
in which
R represents a side chain of a natural or synthetic amino acid,
R₁ represents either a linear or branched (C₁-C₈)alkyl group, or a linear or branched (C₁-C₈)alkoxy group, or an aryl group, or an aryl(C₁-C₄)alkyl group, or an aryloxy group, or a saturated or unsaturated heterocycle,
n=1 or 2 and
A represents an aromatic or heteroaromatic group with one or more rings.

17. Method for preparing a material for thermal insulation comprising an aerogel said method comprising the following steps:
a. preparing an organogel from the pseudopeptides of formula (I). 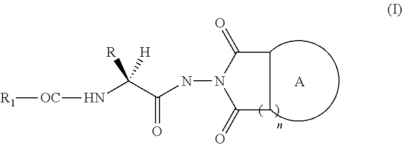
in which
R represents a side chain of a natural or synthetic amino acid,
R₁ represents either a linear or branched (C₁-C₈)alkyl group, i.e. a linear or branched (C₁-C₄)alkoxy group, or an aryl group, or an aryl(C₁-C₄)alkyl group, or an aryloxy group, or a saturated or unsaturated heterocycle,
n=1 or 2 and
A represents an aromatic or heteroaromatic group with one or more rings.
b. drying the thus obtained organogel.

18. Material for thermal insulation according to claim 10, characterized in that the pseudopeptide of formula (I) is chosen from those in which R represents either —CH₂Ph, or —CH(CH₃)₃ or —CH₂CH(CH₃)₃ and R₁ represents either PhCH₂OCO—, or CH₂═CH—CH₂OCO—.
